(19) United States
(12) Patent Application Publication
(10) Pub. No.: US 2022/0225893 A1
(43) Pub. Date: Jul. 21, 2022

(54) METHODS FOR AUTOMATIC PATIENT TIDAL VOLUME DETERMINATION USING NON-CONTACT PATIENT MONITORING SYSTEMS

(71) Applicant: Covidien LP, Mansfield, MA (US)

(72) Inventors: Cynthia A. MILLER, Laguna Hills, CA (US); Paul S. ADDISON, Scotland (GB)

(21) Appl. No.: 17/573,315

(22) Filed: Jan. 11, 2022

Related U.S. Application Data
(60) Provisional application No. 63/137,866, filed on Jan. 15, 2021.

(51) Int. Cl. A61B 5/091; A61B 5/00; A61B 5/1171

(52) U.S. Cl. CPC A61B 5/091; A61B 5/4869; A61B 5/0002; A61B 5/1176; A61B 5/0077

(57) ABSTRACT

Methods and systems for determining patient tidal volume using video-based non-contact patient monitoring technology generally include using a video-based non-contact patient monitoring system, such as a depth sensing camera, to determine one or more characteristics of a patient, and using the measured characteristics when calculating tidal volume. In some embodiments, the non-contact patient monitoring system is used to determine a patient's height, which is then used to calculate a predictive body weight (PBW) of the patient. The calculated PBW can then be used to calculate a patient tidal volume, and the calculated patient tidal volume can be used for ventilator settings. In other embodiments, non-contact patient monitoring systems are used to determine one or more of the length of a one or more segments of patient's body, a patient's gender, and a patient's body volume, each of which can then be used in various ways to calculate patient tidal volume.

### CROSS-REFERENCE TO RELATED APPLICATIONS

The present application claims priority under 35 U.S.C. § 119(e) to U.S. Provisional Patent Application No. 63/137,886, entitled “Methods for Automatic Patient Tidal Volume Determination Using Non-Contact Patient Monitoring Systems”, filed Jan. 15, 2021, the entirety of which is incorporated herein by reference.

### TECHNICAL FIELD

The present disclosure relates to the use of non-contact patient monitoring systems to automatically determine a patient tidal volume for breathing. In some embodiments, the systems and methods described herein can employ non-contact patient monitoring technology to determine various characteristics of a patient that can then be used in calculating an appropriate patient tidal volume. Non-limiting examples of patient characteristics that can be obtained using non-contact patient monitoring technology include patient height, patient gender, and the length of one or more segments of the patient's body. In some embodiments, the measured patient characteristic or characteristics obtained using non-contact patient monitoring technology are used to calculate predicted (also sometimes referred to as ideal) patient height and/or body weight, which are then used to calculate an appropriate patient tidal volume.

### BACKGROUND

The outcome of mechanical ventilation may be influenced by the size of breath given to a patient in relation to the size of that patient's lungs. The size of the lungs is influenced by, e.g., the height and gender of the patient, which in turn determines ideal/predicted body weight. Lung protection ventilation strategies are based on keeping delivered volume within a target range of mL of volume delivered for each kg of ideal/predicted body weight (mL/kg).

When a ventilator is used on a patient, various initial settings are input to ensure that the amount of air supplied to the ventilated patient with each breath is appropriate for the size of that patient's lungs. Initial tidal volume-related settings can be selected using patient demographics, such as gender, height, and/or predicted (ideal) weight of the patient. In one example, a predicted body weight (PBW) is calculated based on the gender and height of the patient using one of various preestablished formula (see, e.g., Moreault, O., Lacasse, Y., & Bussières, J. S. (2017). Calculating ideal body weight: Keep it simple. Anesthesiology: The Journal of the American Society of Anesthesiologists, 127(1), 203-204). The PBW measurement is then used to set an initial tidal volume setting on the ventilator, again using one of various preestablished formula or correlation charts.

Selecting an appropriate tidal volume setting for a ventilator can therefore depend heavily on obtaining accurate measurements of the various patient demographics. In an ideal setting, a patient's height is manually measured by a clinician so that subsequent calculations used to determine an appropriate tidal volume setting and which rely on the patient's height are as accurate as possible. However, it has been recently observed that many clinicians continue to only estimate a patient's height based on visual observation. Furthermore, in emergency situations, the clinician may not have the time or ability to take a manual measurement of the patient. As a result, erroneous tidal volume settings occur more frequently than desired based on these inaccurate patient demographic measurements.

Accordingly, a need exists for methods and systems capable of automating an accurate measurement of various patient demographics used in establishing appropriate patient tidal volume so that more appropriate tidal volume settings can be used, regardless of the clinician's ability to manually measure such patient demographics.

### SUMMARY

Described herein are various embodiments of methods and systems for automatic determination of a patient's tidal volume using non-contact video-based patient monitoring technology. In one embodiment, a video-based patient monitoring method includes: obtaining a depth sensing image of a patient using a depth sensing camera, the depth sensing image encompassing at least the length of the patient's body; from the depth sensing image, determining the patient's height; calculating a predictive body weight of the patient based on the determined patient height; and calculating a tidal volume for the patient based on the calculated predictive body weight.

In another embodiment, a video-based patient monitoring method includes: obtaining a depth sensing image of a patient using a depth sensing camera; from the depth sensing image, determining the length of a segment of the patient's body; calculating a patient height from the length of the segment of the patient's body; calculating a predicted body weight of the patient based on the calculated patient height; and calculating a tidal volume for the patient based on the calculated predicted body weight.

In another embodiment, a video-based patient monitoring method includes: obtaining a depth sensing image of a patient using a depth sensing camera, the depth sensing image encompassing at least the patient's body; from the depth sensing image, determining the patient's body volume; and calculating a tidal volume of the patient based on the patient's body volume.

### BRIEF DESCRIPTION OF THE DRAWINGS

Many aspects of the present disclosure can be better understood with reference to the following drawings. The components in the drawing are not necessarily to scale. Instead, emphasis is placed on illustrating clearly the principles of the present disclosure. The drawings should not be taken to limit the disclosure to the specific embodiments depicted but are for explanation and understanding only.

FIG. 1 is a schematic view of a video-based patient monitoring system configured in accordance with various embodiments of the present technology.

FIG. 2 is a block diagram illustrating a video-based patient monitoring system having a computing device, a server, and one or more image capturing devices, and configured in accordance with various embodiments of the present technology.

FIG. 3 is flow chart illustrating a patient monitoring method configured in accordance with various embodiments of the present technology

FIG. 4 is an illustration of a non-contact video-based patient monitoring system suitable for use in various embodiments of the present technology.

FIG. 4A is an illustration of a non-contact video-based patient monitoring system suitable for use in various embodiments of the present technology.

FIG. 5 is an illustration of a method for determining a ratio of patient shoulder to patient waist suitable for use in embodiments of the present technology.

FIG. 6 is an illustration of a method for determining patient height suitable for use in various embodiments of the present technology.

FIG. 7 is an illustration of a method for determining patient height suitable for use in various embodiments of the present technology.

FIG. 8 is an illustration of a method for determining patient tidal volume suitable for use in various embodiments of the present technology.

### DETAILED DESCRIPTION

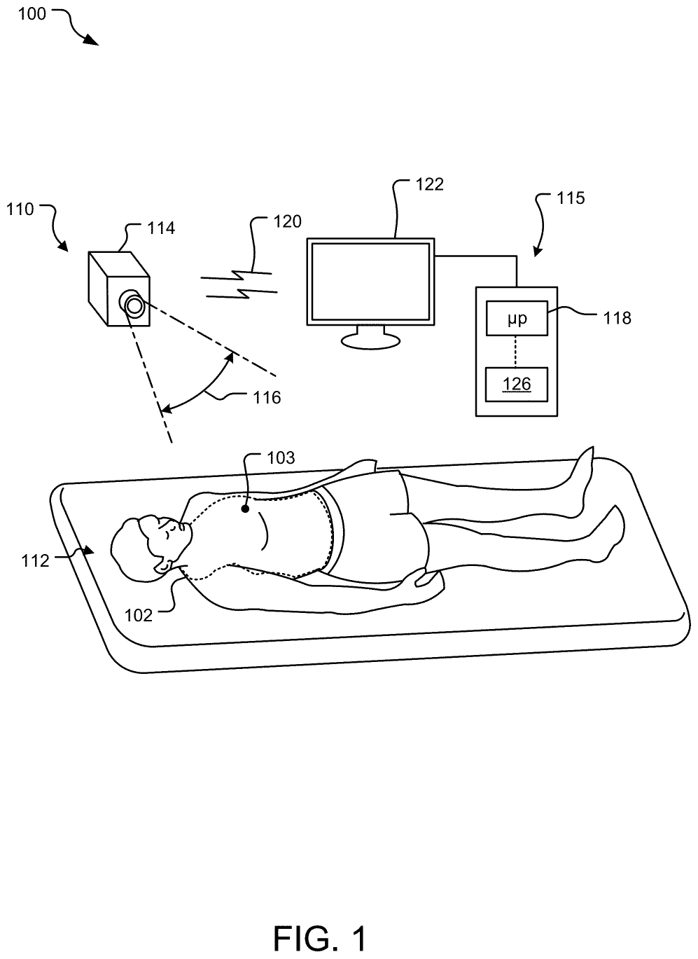

Specific details of several embodiment of the present technology are described herein with reference to FIGS. 1-8. Although many of the embodiments are described with respect to devices, systems, and methods for automatic determination of patient tidal volume using video-based non-contact patient monitoring technology, other applications and other embodiments in addition to those described herein are within the scope of the present technology. For example, at least some embodiments of the present technology can be useful for video-based monitoring of non-patients (e.g., elderly or neonatal individuals within their homes). It should be noted that other embodiments in addition to those disclosed herein are within the scope of the present technology. Further, embodiments of the present technology can have different configurations, components, and/or procedures than those shown or described herein. Moreover, a person of ordinary skill in the art will understand that embodiments of the present technology can have configurations, components, and/or procedures in addition to those shown or described herein and that these and other embodiments can be without several of the configurations, components, and/or procedures shown or described herein without deviating from the present technology.

FIG. 1 is a schematic view of a patient 112 and a video-based patient monitoring system 100 configured in accordance with various embodiments of the present technology. The system 100 includes a non-contact detector 110 and a computing device 115. In some embodiments, the detector 110 can include one or more image capture devices, such as one or more video cameras. In the illustrated embodiment, the non-contact detector 110 includes a video camera 114. The non-contact detector 110 of the system 100 is placed remote from the patient 112. More specifically, the video camera 114 of the non-contact detector 110 is positioned remote from the patient 112 in that it is spaced apart from and does not contact the patient 112. The camera 114 includes a detector exposed to a field of view (FOV) 116 that encompasses at least a portion of the patient 112.

The camera 114 can capture a sequence of images over time. The camera 114 can be a depth sensing camera, such as a Kinect camera from Microsoft Corp. (Redmond, Wash.) or Intel camera such as the D415, D435, and SR305 cameras from Intel Corp, (Santa Clara, Calif.). A depth sensing camera can detect a distance between the camera and objects within its field of view. Such information can be used to determine that a patient 112 is within the FOV 116 of the camera 114 and/or to determine one or more regions of interest (ROI) to monitor on the patient 112. Once a ROI is identified, the ROI can be monitored over time, and the changes in depth of regions (e.g., pixels) within the ROI 102 can represent movements of the patient 112.

In some embodiments, the system 100 determines a skeleton-like outline of the patient 112 to identify a point or points from which to extrapolate a ROI. For example, a skeleton-like outline can be used to find a center point of a chest, shoulder points, waist points, and/or any other points on a body of the patient 112. These points can be used to determine one or more ROIs. For example, a ROI 102 can be defined by filling in area around a center point 103 of the chest, as shown in FIG. 1. Certain determined points can define an outer edge of the ROI 102, such as shoulder points. In other embodiments, instead of using a skeleton, other points are used to establish a ROI. For example, a face can be recognized, and a chest area inferred in proportion and spatial relation to the face. In other embodiments, a reference point of a patient's chest can be obtained (e.g., through a previous 3-D scan of the patient), and the reference point can be registered with a current 3-D scan of the patient. In these and other embodiments, the system 100 can define a ROI around a point using parts of the patient 112 that are within a range of depths from the camera 114. In other words, once the system 100 determines a point from which to extrapolate a ROI, the system 100 can utilize depth information from the depth sensing camera 114 to fill out the ROI. For example, if the point 103 on the chest is selected, parts of the patient 112 around the point 103 that are a similar depth from the camera 114 as the point 103 are used to determine the ROI 102.

In another example, the patient 112 can wear specially configured clothing (not shown) that includes one or more features to indicate points on the body of the patient 112, such as the patient's shoulders and/or the center of the patient's chest. The one or more features can include visually encoded message (e.g., bar code, QR code, etc.), and/or brightly colored shapes that contrast with the rest of the patient's clothing. In these and other embodiments, the one or more features can include one or more sensors that are configured to indicate their positions by transmitting light or other information to the camera 114. In these and still other embodiments, the one or more features can include a grid or another identifiable pattern to aid the system 100 in recognizing the patient 112 and/or the patient's movement. In some embodiments, the one or more features can be stuck on the clothing using a fastening mechanism such as adhesive, a pin, etc. For example, a small sticker can be placed on a patient's shoulders and/or on the center of the patient's chest that can be easily identified within an image captured by the camera 114. The system 100 can recognize the one or more features on the patient's clothing to identify specific points on the body of the patient 112. In turn, the system 100 can use these points to recognize the patient 112 and/or to define a ROI.

In some embodiments, the system 100 can receive user input to identify a starting point for defining a ROI. For example, an image can be reproduced on a display 122 of the system 100, allowing a user of the system 100 to select a patient 112 for monitoring (which can be helpful where multiple objects are within the FOV 116 of the camera 114) and/or allowing the user to select a point on the patient 112 from which a ROI can be determined (such as the point 103 on the chest of the patient 112). In other embodiments, other methods for identifying a patient 112, identifying points on the patient 112, and/or defining one or more ROI's can be used.

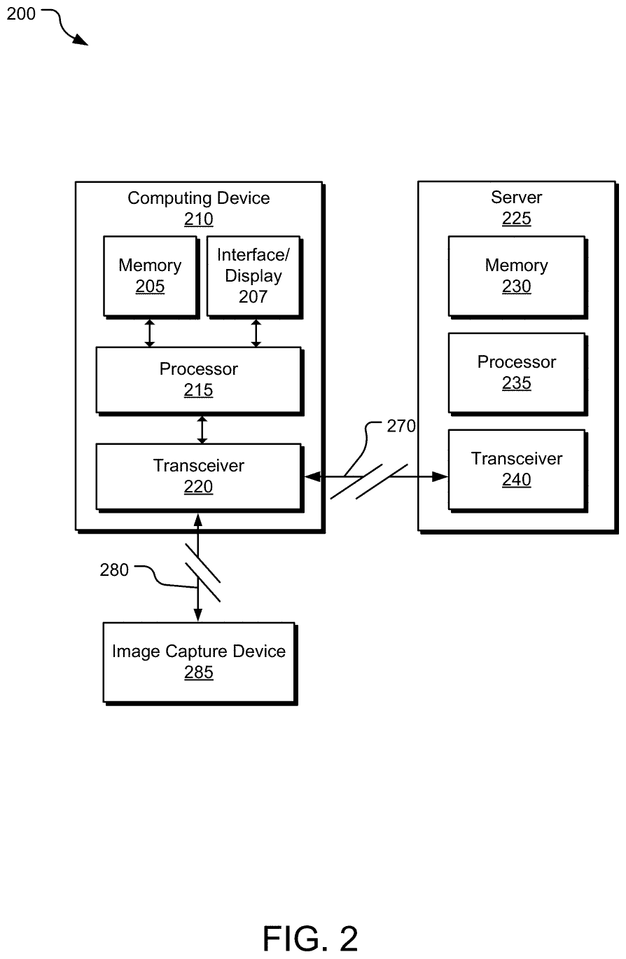

The images detected by the camera 114 can be sent to the computing device 115 through a wired or wireless connection 120. The computing device 115 can include a processor 118 (e.g., a microprocessor), the display 122, and/or hardware memory 126 for storing software and computer instructions. Sequential image frames of the patient 112 are recorded by the video camera 114 and sent to the processor 118 for analysis. The display 122 can be remote from the camera 114, such as a video screen positioned separately from the processor 118 and the memory 126. Other embodiments of the computing device 115 can have different, fewer, or additional components than shown in FIG. 1. In some embodiments, the computing device 115 can be a server. In other embodiments, the computing device 115 of FIG. 1 can be additionally connected to a server (e.g., as shown in FIG. 2 and discussed in greater detail below). The captured images/video can be processed or analyzed at the computing device 115 and/or a server to determine, e.g., a patient's position while lying in bed or a patient's change from a first position to second position while lying in bed. In some embodiments, some or all of the processing may be performed by the camera, such as by a processor integrated into the camera or when some or all of the computing device 115 is incorporated into the camera.

FIG. 2 is a block diagram illustrating a video-based patient monitoring system 200 (e.g., the video-based patient monitoring system 100 shown in FIG. 1) having a computing device 210, a server 225, and one or more image capture devices 285, and configured in accordance with various embodiments of the present technology. In various embodiments, fewer, additional, and/or different components can be used in the system 200. The computing device 210 includes a processor 215 that is coupled to a memory 205. The processor 215 can store and recall data and applications in the memory 205, including applications that process information and send commands/signals according to any of the methods disclosed herein. The processor 215 can also (i) display objects, applications, data, etc. on an interface/display 207 and/or (ii) receive inputs through the interface/display 207. As shown, the processor 215 is also coupled to a transceiver 220.

The computing device 210 can communicate with other devices, such as the server 225 and/or the image capture device(s) 285 via (e.g., wired or wireless) connections 270 and/or 280, respectively. For example, the computing device 210 can send to the server 225 information determined about a patient from images captured by the image capture device(s) 285. The computing device 210 can be the computing device 115 of FIG. 1. Accordingly, the computing device 210 can be located remotely from the image capture device(s) 285, or it can be local and close to the image capture device(s) 285 (e.g., in the same room). In various embodiments disclosed herein, the processor 215 of the computing device 210 can perform the steps disclosed herein. In other embodiments, the steps can be performed on a processor 235 of the server 225. In some embodiments, the various steps and methods disclosed herein can be performed by both of the processors 215 and 235. In some embodiments, certain steps can be performed by the processor 215 while others are performed by the processor 235. In some embodiments, information determined by the processor 215 can be sent to the server 225 for storage and/or further processing.

In some embodiments, the image capture device(s) 285 are remote sensing device(s), such as depth sensing video camera(s), as described above with respect to FIG. 1. In some embodiments, the image capture device(s) 285 can be or include some other type(s) of device(s), such as proximity sensors or proximity sensor arrays, heat or infrared sensors/cameras, sound/acoustic or radio wave emitters/detectors, or other devices that include a field of view and can be used to monitor the location and/or characteristics of a patient or a region of interest (ROI) on the patient. Body imaging technology can also be utilized according to the methods disclosed herein. For example, backscatter x-ray or millimeter wave scanning technology can be utilized to scan a patient, which can be used to define and/or monitor a ROI. Advantageously, such technologies can be able to “see” through clothing, bedding, or other materials while giving an accurate representation of the patient's skin. This can allow for more accurate measurements, particularly if the patient is wearing baggy clothing or is under bedding. The image capture device(s) 285 can be described as local because they are relatively close in proximity to a patient such that at least a part of a patient is within the field of view of the image capture device(s) 285. In some embodiments, the image capture device(s) 285 can be adjustable to ensure that the patient is captured in the field of view. For example, the image capture device(s) 285 can be physically movable, can have a changeable orientation (such as by rotating or panning), and/or can be capable of changing a focus, zoom, or other characteristic to allow the image capture device(s) 285 to adequately capture images of a patient and/or a ROI of the patient. In various embodiments, for example, the image capture device(s) 285 can focus on a ROI, zoom in on the ROI, center the ROI within a field of view by moving the image capture device(s) 285, or otherwise adjust the field of view to allow for better and/or more accurate tracking/measurement of the ROI.

The server 225 includes a processor 235 that is coupled to a memory 230. The processor 235 can store and recall data and applications in the memory 230. The processor 235 is also coupled to a transceiver 240. In some embodiments, the processor 235, and subsequently the server 225, can communicate with other devices, such as the computing device 210 through the connection 270.

The devices shown in the illustrative embodiment can be utilized in various ways. For example, either the connections 270 or 280 can be varied. Either of the connections 270 and 280 can be a hard-wired connection. A hard-wired connection can involve connecting the devices through a USB (universal serial bus) port, serial port, parallel port, or other type of wired connection that can facilitate the transfer of data and information between a processor of a device and a second processor of a second device. In another embodiment, either of the connections 270 and 280 can be a dock where one device can plug into another device. In other embodiments, either of the connections 270 and 280 can be a wireless connection. These connections can take the form of any sort of wireless connection, including, but not limited to, Bluetooth connectivity, Wi-Fi connectivity, infrared, visible light, radio frequency (RF) signals, or other wireless protocols/methods. For example, other possible modes of wireless communication can include near-field communications, such as passive radio-frequency identification (RFID) and active RFID technologies. RFID and similar near-field communications can allow the various devices to communicate in short range when they are placed proximate to one another. In yet another embodiment, the various devices can connect through an internet (or other network) connection. That is, either of the connections 270 and 280 can represent several different computing devices and network components that allow the various devices to communicate through the internet, either through a hard-wired or wireless connection. Either of the connections 270 and 280 can also be a combination of several modes of connection.

The configuration of the devices in FIG. 2 is merely one physical system 200 on which the disclosed embodiments can be executed. Other configurations of the devices shown can exist to practice the disclosed embodiments. Further, configurations of additional or fewer devices than the devices shown in FIG. 2 can exist to practice the disclosed embodiments. Additionally, the devices shown in FIG. 2 can be combined to allow for fewer devices than shown or can be separated such that more than the three devices exist in a system. It will be appreciated that many various combinations of computing devices can execute the methods and systems disclosed herein. Examples of such computing devices can include other types of medical devices and sensors, infrared cameras/detectors, night vision cameras/detectors, other types of cameras, augmented reality goggles, virtual reality goggles, mixed reality goggle, radio frequency transmitters/receivers, smart phones, personal computers, servers, laptop computers, tablets, blackberries, RFID enabled devices, smart watch or wearables, or any combinations of such devices.

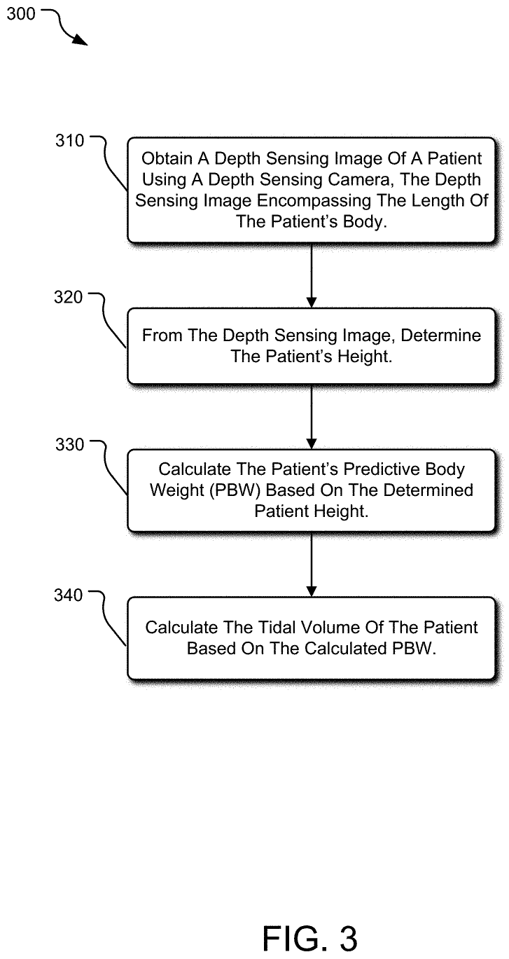

With reference to FIG. 3, a method 300 for use in automatically determining the tidal volume of a patient includes a step 310 of obtaining a depth sensing image of a patient using a depth sensing camera, a step 320 of determining the patient's height from the depth sensing image, a step 330 of calculating the patient's predictive body weight (PBW) using the height measurement obtained in step 320, and a step 340 of calculating the patient's tidal volume using the PBW calculated in step 330. By employing method 300, a clinician can obtain more accurate measurements of the patient tidal volume without having to either manually measure the height of the patient or make an estimate of the patient height based on visual inspection. Instead, the depth sensing camera and associated components of a non-contact patient monitoring system obtain an accurate measurement of the patient's height and automatically perform all subsequent calculations required to provide a tidal volume calculation for the patient. The patient monitoring system may also be communicatively coupled with a ventilator and associated control componentry such that the calculated tidal volume is automatically transmitted to the ventilator and set as the tidal volume for the ventilated patient.

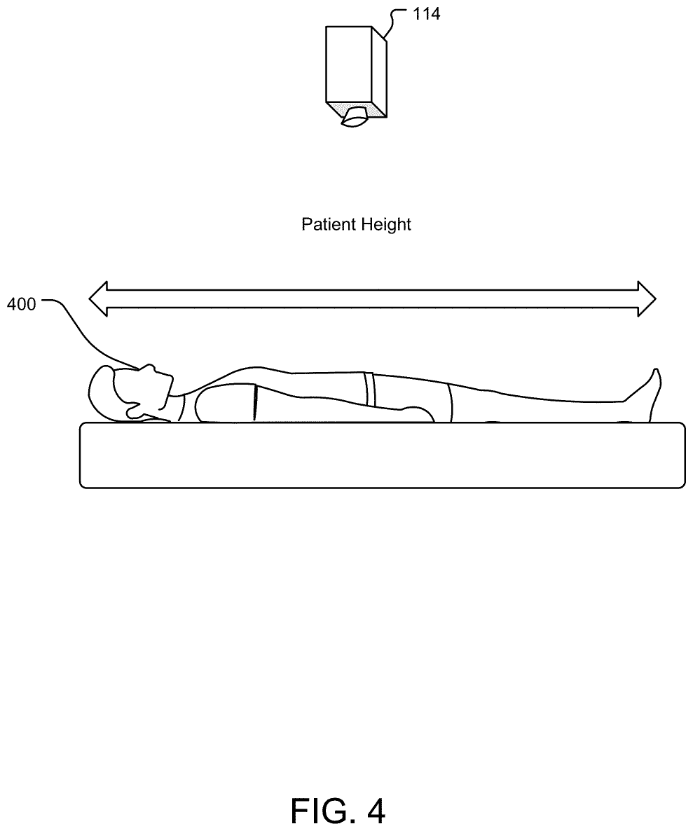

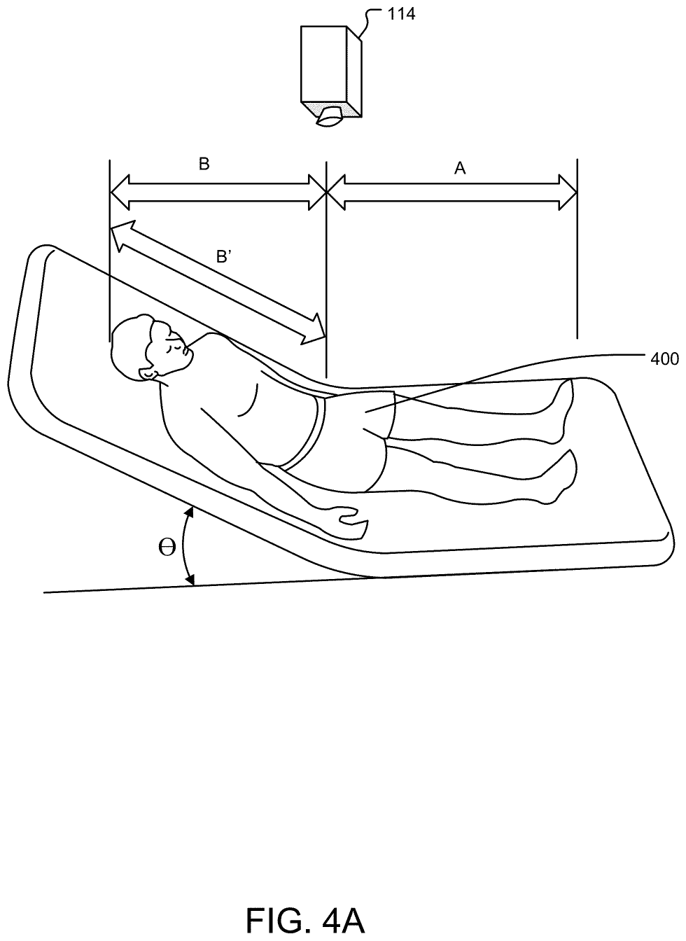

FIGS. 4 and 4A provide further detail regarding steps 310 and 320 of method 300 wherein a depth sensing camera 114 is used to obtain a depth sensing image of the patient 400 and a patient height calculation is extracted from the depth sensing image. Starting with reference to FIG. 4, the camera 114 may be similar or identical to the camera 114 described previously with respect to FIG. 2, and may be positioned over patient 400 so as to have a clear view of the patient 400. While camera 114 is shown in FIG. 4 as being positioned directly over the patient 400, it should be appreciated that the camera 114 can be located at other positions relative to the patient 400, provided that the camera 114 has a clear view of the patient 400. In some embodiments, the camera 114 is positioned so that the depth sensing image of the patient 400 captured by the camera 114 encompasses the length of the patient 400 (i.e., from the patient's head to the patient's feet). In this manner, an accurate height measurement of the patient 400 can be obtained from the depth sensing image. While depth sensing images of the patient 400 including less than the entire length of the patient's body can also be used in the method 300, such instances will generally require that extrapolations and/or assumptions regarding the total height of the patient 400 be made from the measured portion of the patient 400 included in the image, which may then reduce the accuracy of the overall height measurement and subsequent calculations made thereon.

Any manner of determining the patient's height from the depth sensing image captured by the camera 114 can be used. The computing device 115 associated with the camera 114 as shown in FIG. 2 (including processor 118 and memory 124) may include computer-executable instructions specifically designed to analyze and process data within the captured depth sensing image to determine patient height. In some embodiments, the computing device 115 may be programmed with instructions that are capable of using the depth sensing image and associated data to identify the approximate boundary of the patient's body, from which the patient height can then be measured. For example, when the outline of the patient body is determined from the depth sensing image, the software can further identify the head and feet of the patient 400 based on the outline, and measure the distance between the feet and the head to thereby obtain the patient height.

In other embodiments, the computing device 115 runs executable instructions that identify the opposite ends of the patient 400 based on the depth sensing data within the depth sensing image. For example, the computing device 115 may analyze the depth sensing data within the depth sensing image to identify a first end of the patient 400 and a second end of the patient 400 opposite the first end. Identifying these ends may be based on, e.g., identifying locations within the depth sensing image where relatively large changes in measured distance occur, such large changes denoting a transition from the patient's body to the bed upon which the patient 400 is positioned.

The computing device 115 may also employ, in conjunction with the camera 114 and the captured patient depth sensing image, computer-executable instructions capable of identifying a predicted patient foot region and/or a predicted patient head region within the depth sensing image. Such predicted regions can then be used to measure the height of the patient 400.

With respect to identifying a predicted foot region of the patient 400, a predicted foot region can be determined using any suitable methods, including using methods similar to those described previously. For example, in a method where the depth sensing image is used to determine an outline of the patient, the shape of the obtained outline can be analyzed to identify the predicted foot region. The predicted foot region can be identified based on the predicted foot region having a shape similar to an expected foot region for standard body outlines, or may be identified based on its spatial relation to other portions of the outline that are identified as other parts of the patient's body. For example, identification of a hand, waist, hip, etc., from the obtained boundary shape of the patient may then be used to identify other portions of the outline based on proximity/spatial relation to the identified body part.

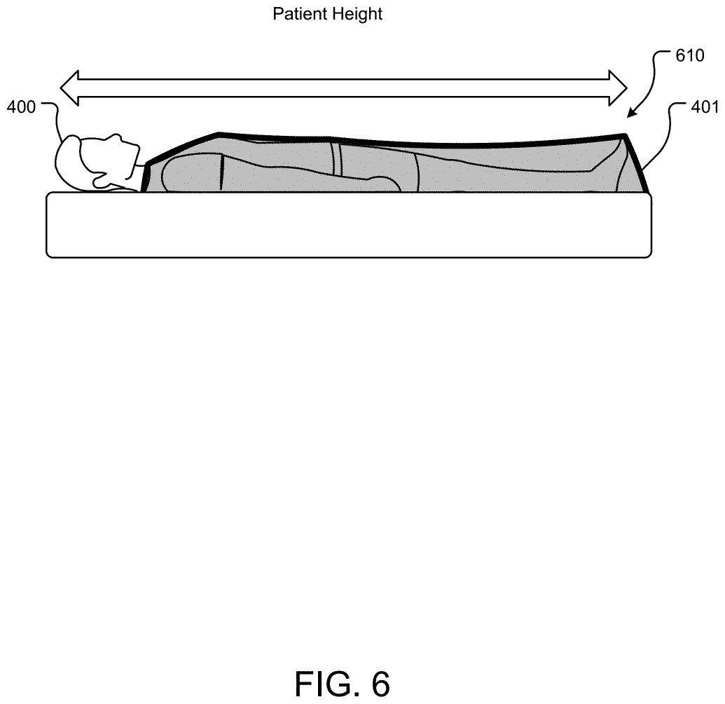

With reference to FIG. 6, identifying a first end of the patient 400 (i.e., the feet of the patient 400) for purposes of measuring the height of the patient 400 may be complicated by the presence of a blanket covering a portion of the patient's body. As shown in FIG. 6, patient 400 is covered from the neck down by a blanket 401. The presence of the blanket 401 may result in some amount of draping at different portions of the patient's body and/or obscure the outline of the patient's body, which may impact depth readings in the associated depth image captured by a depth sensing camera positioned over the patient 400. In order to accommodate for these problems, a method of identifying a first end of a patient 400 for purposes of ultimately determining a patient's height may include identifying a predicted foot region, such as by any of the method described previously, and then identifying within the predicted foot region a peak height 610. The peak height 610 within the predicted foot region is then assumed to be the toes of the patient 400, which then allows for identifying the first end of the of the patient 400 at or proximate the peak height 610.

With respect to identifying a predicted head region of the patient 400, a predicted head region can be determined using any suitable method, including using methods similar to those described previously with respect to identifying a predicted foot region. In some embodiments, facial recognition software can be integrated into the computing device 115 so that the facial recognition software can be used to assist with identifying a predicted head region. For example, facial recognition software can be used to identify the location of a face in the image captured by the camera 114. Location of a face by facial recognition software can then allow for assigning a predicted head region in the location of and/or encompassing the recognized face. A more precise predicted head region determination can be achieved by further using the identification of specific facial features within a facial recognition. For example, identification of a nose or eyes within a facial recognition analysis can then be used to more accurately identify a predicted head region, the predicted head region being established at least based on its proximity and/or special relation to the identified facial features.

With reference to FIG. 4A, the methods and systems described herein may also take into account whether a portion of the patient 400 is positioned at an angle to thereby provide a more accurate height measurement. As shown in FIG. 4A, the patient's torso is at an angle based on the upper portion of the bed being positioned at an angle. If the angle at which the patient is propped is not taken into account, the height of the patient 400 may be calculated as the sum of segments A and B shown in FIG. 4A. However, such calculation would underestimate the height of the patient 400. Accordingly, the method 300 outlined in FIG. 3 may include an additional step in which prior to the calculation of the patient height, it is first determined if the patient or portion of the patient is positioned at an angle. Any suitable method for determining whether the patient or a portion of the patient is positioned at an angle may be used. In some embodiments, the bed on which the patient 400 is positioned may be communicatively coupled with the patient monitoring system such that any degree to which the bed is angled is communicated to the computing device 115 for consideration in subsequent calculations regarding patient height. In other embodiments, the depth sensing image is used to determine whether the patient is positioned at an angle. For example, if a torso of the patient 400 is positioned at an angle, the depth measurements to the patient's head will be much smaller than the depth measurements to the patient's legs. Such a discrepancy can serve as an initial indicator that the patient is not lying flat on his or her back.

When the system identifies the patient or a portion of the patient as being positioned at an angle, the calculation of the patient's height can be adjusted to take into account such an angle. Any suitable manner of calculating the patient's height as being the sum of segments A and B′ shown in FIG. 4A rather than the sum of segments A and B can be used. In some embodiments, such as embodiments where the angle Θ is known or can be calculated, basic trigonometry can be used to determine the length of B′. For example, B′ may be calculated as B/cos Θ when the value of both B and Θ can be obtained from the non-contact patent monitoring system. Pythagorean's theorem can also be used to determine B′ when B and the height the patient's head is away from the bed are known (both of which information may be obtained from the depth sensing camera and the depth sensing image).

Once the patient's height has been determined in step 320, such as by using any of the methods described previously, the method 300 proceeds to step 330, wherein a predictive body weight (PBW) is calculated based on the height determined in step 320. Any known formula or correlation chart used to calculate PBW from height can be used to carry out step 330. One exemplary formula is described in Moreault, O., Lacasse, Y., & Bussières, J. S. (2017). Calculating ideal body weight: Keep it simple. Anesthesiology: The Journal of the American Society of Anesthesiologists, 127(1), 203-204. For example, a man's predicted body weight can be calculated based on measured height using the following formula:

Weight (kg)=50 kg+(0.91×[Patient Height in Centimeters−152.5])

As noted previously, this calculation can be carried out automatically using the computing device 115. In some embodiments, the computing device 115 is used to determine the patient height in step 320 as described previously, and therefore the computing device 115 immediately has possession of the height calculation for implementation into the PBW calculation in step 330.

As alluded to previously, calculation of PBW may depend on the gender of the patient. For example, the formula provided previously provides a means for determining a PBW based on patient height for when the patient is a man. The formula for a woman is different, and therefore it may be beneficial for the methods described herein to further take into account the gender of the patient when calculating PBW. In some embodiments, the gender of the patient can be manually input into the system so that the appropriate formula is used when carrying out step 330. However, some embodiments of the method may include an additional step wherein the gender of the patient is determined using the non-contact patient monitoring system.

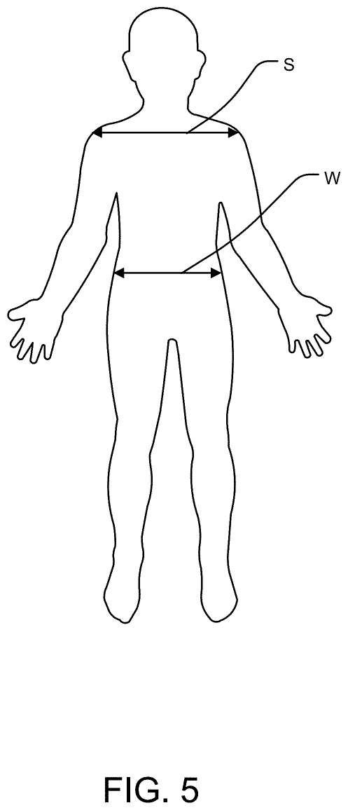

Any suitable method for determining patient gender using non-contact patient monitoring systems can be used. In some embodiments, patient gender is determined by calculating a ratio of shoulder length (S) to waist length (W). With reference to FIG. 5, both the measurement S and W can be obtained using the depth sensing technology previously described herein. For example, the depth sensing camera can obtain a depth sensing image of a patient, within which the patient's shoulders and waist can be identified. For example, methods described previously for identifying various parts of the patient's body can be used to identify the patient's shoulders and waist. Once identified, the length of these body parts can be measured to calculate an S:W ratio. Once determined, the S:W ratio can be compared against previously tabulated data regarding typical S:W ratio values for men and women to thus determine a predicted gender of the patient. Once determined, subsequent calculation of the patient's PBW in step 330 is adjusted to use the appropriate formula for a man or a woman. Any other suitable body ratios or body segment lengths known to be useful in predicting patient gender can also be used.

In step 340, the predictive body weight obtained in step 330 is used to calculate the patient's tidal volume. Any known formula or correlation chart used to calculate tidal volume from PBW can be used. In some embodiments, the tidal volume (in mL) is calculated as being 4 to 10 times the PBW of the patient in kilograms. In other words, the tidal volume used for the ventilator setting is set as being 4 to 10 mL per kilogram of the patient. As with step 330 discussed above, this calculation can be carried out automatically using the computing device 115. In embodiments where the computing device 115 is used to automatically calculate the PBW from the patient height and gender determined automatically by the non-contact patient monitoring technology associated with the computing device 115, the computing device 115 is also immediately automatically apply this value to the tidal volume calculation to immediately obtain the desired tidal volume setting for the ventilator.

In embodiments where the tidal volume is calculated as being 4 to 10 times the PBW of the patient in kilograms, it may be necessary to select the specific value between 4 and 10 that is used to carry out the tidal volume calculation. The specific value between 4 and 10 is often selected based on facility and/or clinician preference. Thus, in some embodiments, it may be possible for the preferred value to be entered into and stored in the computing device 115 such that the preferred value between 4 and 10 is automatically used when calculating tidal volume. In a scenario where different clinicians within the same facility and using the same equipment have a different preference for the value between 4 and 10 to be used when calculating tidal volume, it is possible for the unique preference of each clinician to be entered into and stored in the computing device 115 and for the computing device 115 to recognize and/or be told which clinician is treating the monitored patient such that each clinician's preferred value is automatically applied when calculating tidal volume.

Once the tidal volume calculation is determined in step 340, a ventilator's tidal volume setting can be programmed for a specific patient based on the tidal volume calculated in step 340. When the ventilator is communicatively associated with, for example, the computing device 115, the calculated tidal volume value can be automatically and immediately sent to the ventilator for use in initial ventilator settings for the patient.

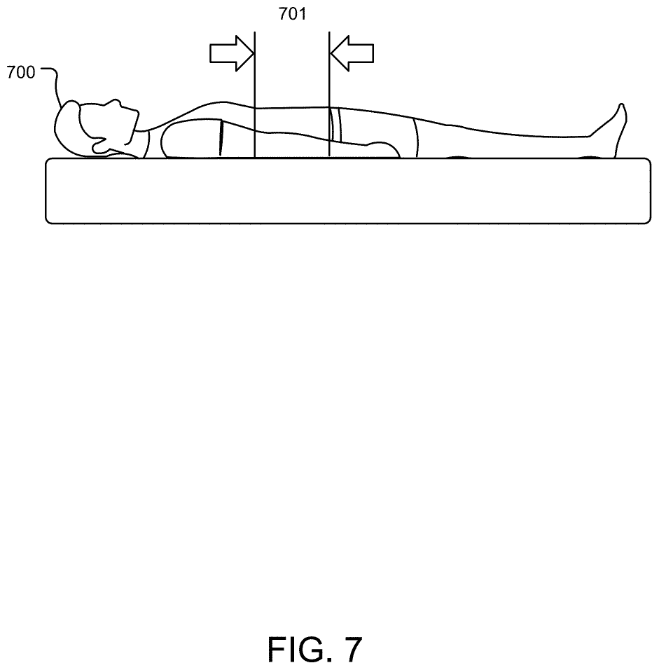

Method 300 described previously generally relates to measuring a patient's height and determining patient tidal volume from the measured patient height (via a PBW calculation that depends on the measured patient height). However, it should be appreciated that modifications to method 300 can be implemented such that the patient's height is calculated from measuring a segment of the patient's body, rather than directly measuring the patient's overall height. With reference to FIG. 7, once such modification involves measuring the length of a patient's ulna 701 and using the measured length of the patient's ulna 701 to calculate a predicted height of the patient 700. Any known correlation between ulna length and overall patient length can be used. One example of a correlation between ulna length and patient height that can be used in the methods described herein is set forth in Barbosa, V. M., Stratton, R. J., Lafuente, E. & Elia M. (2012). Ulna length to predict height in English and Portugese patient populations. European journal of clinical nutrition, 66 (2), 209.

It should be appreciated that a modification to method 300 as described previously is not limited to measuring ulna length and calculating patient height from ulna length. Any other body segment that has been correlated to overall body height can be used in this embodiment. Regardless of the body segment selected, the manner of identifying and measuring the selected body segment can be similar or identical to methods described previously with respect to identifying various parts of the patient in a depth sensing image and using non-contact patient monitoring systems.

Once the patient's predicted height is calculated from a patient's body segment, the method 300 can generally progress in the same manner as described previously (i.e., where PBW is calculated from the calculated patient height in step 330 and tidal volume is then calculated from the calculated PBW in step 340).

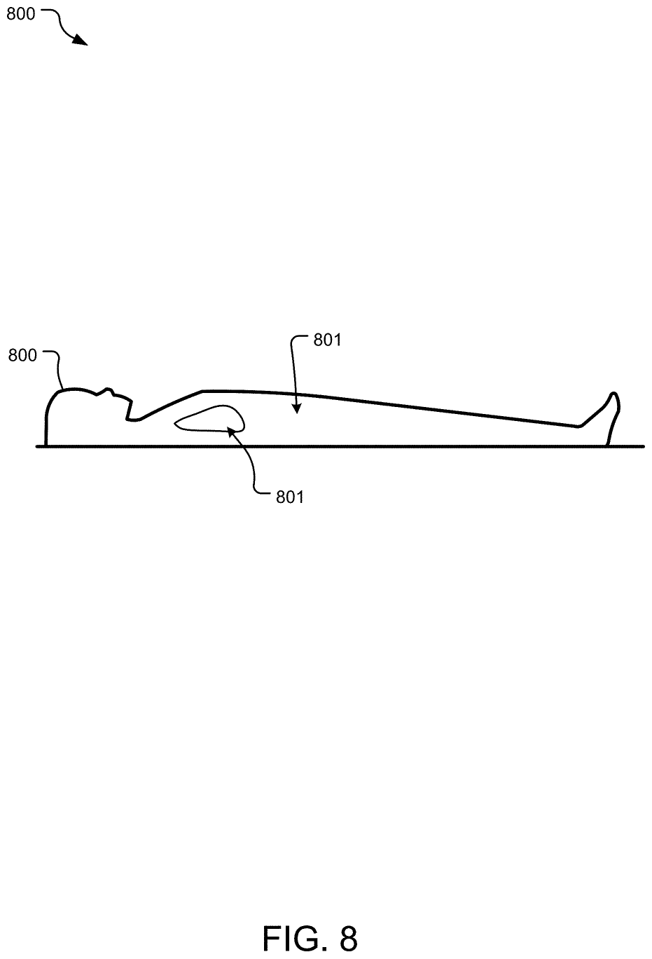

With reference to FIG. 8, other embodiments of the technology described herein may use non-contact patient monitoring technology to determine the total body volume 801 (V_{b}) of a patient 800 and then calculate a patient's tidal volume 802 from the determined total body volume 801 using previously known and established ratios (referred to as Rᵥ) of total body volume to tidal volume (Vₜ) (where Rᵥ=Vₜ/V_{b}). In such embodiments, a depth sensing camera is used to obtain a depth sensing image of the patient's entire body, and the total volume 801 of the patient's body is calculated using the depth sensing data from the depth sensing image. For example, in some embodiments, the total volume 801 of the patient's body can be obtained from the depth sensing image by integrating the depth measurements across the patient's body. In other embodiments, a patient's body is scanned prior to getting into bed to determine a patient total body volume 801. Any other suitable method of obtaining patient total volume 801 can also be used.

Once a patient's total body volume 801 is determined using the depth sensing image or other techniques, the computing device 115 (as shown in FIG. 2). can be used to automatically calculate patient tidal volume 802 by accessing standard and pre-established Rᵥ values. For example, in some embodiments, Rᵥ values are obtained from existing population studies. In some embodiments, the Rᵥ value used for the patient 800 is selected based on other measured characteristics of the patient 800. For example, the Rᵥ value may depend on one or more patient demographics, such as the gender of the patient, the age of the patient, etc. As such, the determination of the patient tidal volume 802 using a measured total patient body volume 801 and an appropriate Rᵥ value may further employ methods of determining certain patient characteristics and or demographics prior to selecting the appropriate Rᵥ value to use in the calculation of patient tidal volume.

In some embodiments, the method further includes a step of determining the gender of the patient 800 prior to selecting the appropriate Rᵥ value. Determination of patient gender may employ similar techniques as described herein previously, such as measuring a patient S:W ratio and making a determination of gender by comparing the calculated S:W ratio to preestablished S:W values associated with men or women. Once the gender is determined, an appropriate Revalue can be selected such that a more accurate tidal volume calculation can be carried out.

Determination of patient age can also be carried out using non-contact patient monitoring technology. In some embodiments, a rough approximation of age (e.g., infant, adolescent, adult) is all that is required to select an appropriate Rᵥ value. In such embodiments, the non-contact patient monitoring technology is used to make a determination of patient age. For example, a depth sensing image captured from a depth sensing camera and which includes an overall outline of the patient body can be used to make determinations of patient age based on, e.g., the overall size of the patient outline and other ratios of body segments that denote whether a patient is an infant, adolescent or adult.

Information on patient gender, age, etc. can also be obtained by other means, such as manually inputting such data into the computing device 115 by a clinician. This data can then be accessed by the computing device 115 at the appropriate time for calculation of the patient tidal volume 802 in conjunction with the measured patient total body volume 801.

From the foregoing, it will be appreciated that specific embodiments of the invention have been described herein for purposes of illustration, but that various modifications may be made without deviating from the scope of the invention. Accordingly, the invention is not limited except as by the appended claims.

I/We claim:

1. A video-based patient monitoring method, comprising:
obtaining a depth sensing image of a patient using a depth sensing camera, the depth sensing image encompassing at least the length of the patient's body;
from the depth sensing image, determining the patient's height;
calculating a predictive body weight of the patient based on the determined patient height; and
calculating a tidal volume for the patient based on the calculated predictive body weight.

2. The method of claim 1, wherein determining the patient's height comprises:
identifying a first end of the patient in the depth sensing image;
identifying a second end of the patient generally opposite the first end of the patient in the depth sensing image; and
measuring the distance between the first end and the second end.

3. The method of claim 2, wherein identifying the first end of the patient comprises:
identifying a predicted foot region within the depth sensing image;
determining a peak height within the predicted foot region; and
assigning the first end of the patient as a location proximate the peak height in the predicted foot region.

4. The method of claim 1, wherein identifying the second end of the patient comprises:
identifying a predicted head region within the depth sensing image; and
assigning the second end of the patient as a location proximate the identified predicted head region.

5. The method of claim 4, wherein facial recognition analysis is used as part of identifying the predicted head region.

6. The method of claim 1, further comprising:
from the depth sensing image, determining if a portion of the patient's body is positioned at an angle; and
when a portion of the patient's body is positioned at an angle, adjusting the determination of the patient's height by taking into account the angled portion of the patient's body.

7. The method of claim 1, further comprising:
from the depth sensing image, predicting the patient's gender; and
calculating the predictive body weight for the patient based on the determined patient height and the patient's predicted gender.

8. The method of claim 7, wherein predicting the patient's gender comprises:
from the depth sensing image, identifying a patient's waist;
measuring the length of patient's waist;
from the depth sensing image, identifying the patient's shoulders;
measuring the length of the patient's shoulders;
calculating a shoulder to waist (S:W) ratio; and
predicting the patient's gender based on the S:W ratio.

9. A video-based patient monitoring method, comprising:
obtaining a depth sensing image of a patient using a depth sensing camera;
from the depth sensing image, determining the length of a segment of the patient's body;
calculating a patient height from the length of the segment of the patient's body;
calculating a predictive body weight of the patient based on the calculated patient height; and
calculating a tidal volume for the patient based on the calculated predictive body weight.

10. The method of claim 9, wherein the segment of the patient's body is the patient's ulna.

11. A video-based patient monitoring method, comprising:
obtaining a depth sensing image of a patient using a depth sensing camera, the depth sensing image encompassing at least the patient's body;
from the depth sensing image, determining the patient's body volume; and
calculating a tidal volume of the patient based on the patient's body volume.

12. The method of claim 11, wherein calculating the tidal volume of the patient is further based on a preestablished ratio of body volume to tidal volume.

13. The method of claim 12, further comprising:
from the depth sensing image, identifying a patient's waist;
measuring the length of patient's waist;
from the depth sensing image, identifying the patient's shoulders;
measuring the length of the patient's shoulders; and
calculating a shoulder to waist (S:W) ratio;
wherein the preestablished ratio of body volume to tidal volume used in calculating the tidal volume of the patient is selected based on the calculated S:W ratio.

14. The method of claim 12, further comprising:
from the depth sensing image, identifying a patient's waist;
measuring the length of patient's waist;
from the depth sensing image, identifying the patient's shoulders;
measuring the length of the patient's shoulders;
calculating a shoulder to waist (S:W) ratio; and
predicting the patient's gender based on the S:W ratio.
wherein the preestablished ratio of body volume to tidal volume used in calculating the tidal volume of the patient is selected based on the predicted gender of the patient.

15. The method of claim 12, further comprising:
from the depth sensing image, determining if the patient is an adult or a child;
wherein the preestablished ratio of body volume to tidal volume used in calculating the tidal volume of the patient is selected based on the determination of whether the patient is an adult or a child.
